(19) United States
(12) Patent Application Publication
(10) Pub. No.: US 2026/0257042 A1
(43) Pub. Date: Sep. 3, 2026

(54) DEVICE FOR REVERSIBLY SECURING A FLEXIBLE CONDUIT TO THE SKIN OF A PATIENT

(71) Applicant: GUY'S AND ST. THOMAS' NHS FOUNDATION TRUST, London, Greater London (GB)

(72) Inventors: Emmanuel AKINLUYI, London, Greater London (GB); Jacob WILLIAMS, London, Greater London (GB); Maia HEATH, London, Greater London (GB); Silke HEYSE, London, Greater London (GB)

(21) Appl. No.: 18/881,702

(22) PCT Filed: Jul. 10, 2023
(86) PCT No.: PCT/GB2023/051813

§ 371 Date: Jan. 6, 2025

(30) Foreign Application Priority Data
Jul. 11, 2022 (GB) 2210151.3

(51) Int. Cl. A61M 25/02

(52) U.S. Cl. CPC A61M 25/02; A61M 2025/026; A61M 2025/028; A61M 2205/0227; A61M 2205/583; A61M 2209/088; A61M 2210/04

(57) ABSTRACT

A device (100) for reversibly securing a flexible conduit to the skin of a patient, includes: a rigid base (110) that is secured to a patient's skin by an adhesive element (120). The base (110) includes a surface for supporting a conduit such as a cannula. The device also includes a flexible strap (130) having a resting curvature, which extends from the base (110). The base (110) and strap (130) have respective interengageable fastening elements (116; 136). The conduit-supporting surface is provided on supports (112) that are themselves spaced from one another. The strap (130) can be fed between the supports (112) so that the fastening element (136) on the strap (130) can access and engage that on base (110). The strap (130) thus, in use, wraps around a conduit, and, once made taut by a clinician, frictionally engages the conduit.

### FIELD OF THE INVENTION

The present invention relates to a device for reversibly securing a flexible conduit to the skin of a patient, and in particular to a device suitable for reversibly securing a cannula to the skin of a patient, for example during extracorporeal membrane oxygenation (ECMO) procedures.

### BACKGROUND OF THE INVENTION

ECMO is a temporary life support technique used to oxygenate the blood of critically ill patients suffering from potentially reversible lung or cardiac failure. In the treatment of these patients, external machinery is employed to replicate the function of the heartbeat and lungs, allowing the patient's impaired organs time to heal. This procedure requires that a pair of cannulae are placed into the large arteries and veins around the heart to remove the patient's blood, which is pumped externally via an artificial lung to be oxygenated, before returning it to the patient's central circulatory system.

The precise position of the tip of each cannula is critical; even a minute shift in the placement of a cannula tip within the body can be fatal to the patient. It is vital therefore that the cannulae are fastened securely where they exit the body. Further complications arise in that treatment by ECMO typically lasts up to six weeks, during which time the patient is awake intermittently and the cannulae may frequently need to be repositioned. During this period, the patient may move or be moved: they might undergo assisted walks for therapeutic exercise, and they may otherwise be positioned in shifting or proning as a measure to prevent the development of pressure sores. These processes place additional stresses on the cannulae.

The prevailing practice is to secure the cannulae to the skin of the patient with sutures, as is shown in FIG. 1, each cannula (C) being sewn to the skin of the patient with stitches (S). Suturing gives rise, however, to a number of problems, both for patients and clinical staff. The sutures need to be replaced frequently (typically every five days) either because they lose purchase or because the cannula tips need to be repositioned. Repeated suturing causes significant pain, scarring, and psychological discomfort to the patient. The ECMO procedure also requires the patient's blood to be thinned, often resulting in continuous bleeding and an associated risk of infection at the suture sites. As well as being traumatic to the patient, the process of installing and replacing the sutures (during which precise control of the placement of the cannulae must be maintained) is onerous and requires multiple clinicians, creating both a risk to the patient and a substantial clinical burden.

Alternative ways of securing cannulae have been developed. However, existing devices suffer a number of shortcomings which make them unsuitable or undesirable for ECMO applications. Broadly, these devices form two categories: cannula clips and cannula ties. Cannula clips typically constitute a rigid clasp. Clasp configurations have been found to require the cannula to be moved out of position during installation and removal of the clip, provide limited adaptability to different insertion sites and sizes of cannulae, and insufficiently prevent crimping of the cannula. Cannula ties typically resemble a cable tie, and provide, by comparison, improved installation and greater adaptability to various sizes of cannulae and areas of the body. However, the degree of fixation and stabilisation that these ties are able to provide is impractically limited, the process of their removal requires undesirable downward pressure, and the possibility of crimping the cannula during their installation remains.

The present invention seeks to provide an improved device for reversibly securing a flexible conduit to the skin of a patient.

### SUMMARY OF THE INVENTION

According to an aspect of the invention, there is provided a device for reversibly securing a flexible conduit to the skin of a patient, the device including: a rigid base having a conduit-supporting portion with a conduit-supporting surface; an adhesive element disposed on an underside of the base to secure the device to the skin; and, a flexible strap extending from the base adjacent to the conduit-supporting portion; wherein: the base has a first fastening element and the strap has a second fastening element, the first and second fastening elements being interengageable with each other; the conduit-supporting portion includes first and second spacers below the conduit-supporting surface; and, the spacers are spaced from one another to receive the strap therebetween, providing the second fastening element access to the first fastening element therethrough.

In embodiments, the strap has a first end attached to the base and a second free end. Preferably, the strap extends from the base at a position above the conduit-supporting portion.

Preferably, the conduit-supporting surface includes first and second portions which are spaced from each other to accommodate the strap therebetween. Further, it is particularly preferred that a respective portion of the conduit-supporting surface is at an upper side of each spacer. In other words, a first portion of the conduit-supporting surface is formed by an upper surface of the first spacer, and a second portion of the conduit-supporting surface is formed by an upper surface of the second spacer.

The base may have a non-solid footprint and may include multiple feet or load-bearing portions. Preferably, the base has three feet. Each foot is preferably positioned at an extremity of the base.

In a preferred embodiment, the first and second spacers form first and second feet of the base.

The first fastening element is preferably positioned on the base at a point higher than or level with the position at which the strap extends from the base.

It is preferred that the strap has multiple fastening elements, each of which is interengageable with the first fastening element on the base.

Preferably, the strap includes a portion having a resting curvature. The strap may have a non-uniform thickness and/or density. For example, the thickness and/or density may vary on one side. A bistable structure may be formed such that the resting curve is activated as the strap is initially deformed.

Preferably, the strap includes a material or surface preparation that exhibits a visual change under specific tensional loads to give a visual indication of over-tightening.

Preferably, the device includes a flexurally-compliant pad disposed between the base and the adhesive element, the pad having a multiplicity of cells.

In embodiments, the base includes an aperture between the first and second spacers, the aperture being configured (or shaped and sized) to receive the strap. In preferred embodiments, the base includes an opening in communication with the aperture, which extends at least from the spacers to the first fastening element.

In embodiments, the strap extends from the base at a position between the conduit-supporting portion and the first fastening element. The strap may extend initially upward from the base.

In embodiments, the first fastening element and the conduit-supporting portion are positioned at opposite ends of the base

The first and second spacers may be connected by a strut, and optionally the strap extends from the strut

The first and second fastening elements are preferable releasably interengageable with each other. The multiple fastening elements of the strap are preferably spaced from one another lengthways on the strap.

In some embodiments, the adhesive element has: a membrane; and, an adhesive disposed on an underside of the membrane. Preferably, the adhesive is disposed on the membrane in a zigzag between first and second ends of the membrane, the first end of the membrane having a pull tab.

According to another aspect of the invention, there is provided a device for reversibly securing a flexible conduit to the skin of a patient, including: a rigid base having a conduit-engaging portion; an adhesive element disposed on an underside of the base to secure the device to the skin; and, a flexible strap extending from the base; wherein the base has a first fastening element and the strap has a second fastening element, the first and second fastening elements being interengageable with each other; and the base has a non-solid footprint and multiple load-bearing portions or feet.

Preferably there are three load-bearing portions or feet.

According to another aspect of the present invention, there is provided a method of reversibly securing a flexible conduit to the skin of a patient including: adhering a device as claimed in any preceding claim to the skin of a patient; supporting a conduit on the conduit-supporting surface; wrapping the strap around the conduit; feeding the strap between the spacers; and engaging the first and second fastening elements.

The method may include removing excess length at the free end of the strap.

Preferred features of all described aspects and embodiments are combinable with other aspects and embodiments.

### BRIEF DESCRIPTION OF THE DRAWINGS

Embodiments of the present invention are described below, by way of example only, with reference to the accompanying drawings, in which:

FIG. 1 shows a cannula inserted and sutured at the groin of a patient undergoing ECMO treatment;

FIG. 2 is perspective view of a device according to an embodiment;

FIG. 3 is a plan view of the device of FIG. 2;

FIG. 4 is a partial front view of the device of FIG. 2;

FIG. 5 shows, in a profile view, the device of FIG. 2 as fastened around a cannula;

FIG. 6 is a perspective view of the fastened device as shown in FIG. 5;

FIG. 7 is a plan view, showing example dimensions of the device of FIG. 2;

FIG. 8 is a representation of the layers of an adhesive element incorporated in the device of FIG. 2;

FIGS. 9 and 10 show the configuration of a membrane comprised within the adhesive element;

FIG. 11 shows the underside of the adhesive element;

FIG. 12 shows a process of placing the device of FIG. 2 adjacent to a conduit (e.g. cannula) and securing the device to a surface, such as the skin of a patient;

FIGS. 13 and 14 show a process of fastening the device of FIG. 2 to a conduit (e.g. cannula) once secured to the surface, such as the skin of a patient;

FIG. 15 shows a process of removing and replacing the device of FIG. 2 once secured and fastened;

FIG. 16 is a perspective view of a device according to another embodiment;

FIG. 17 is a profile view of the device of FIG. 16;

FIG. 18 is a front view of the device of FIG. 17; and

FIG. 19 is a partial perspective view of a further embodiment of the device.

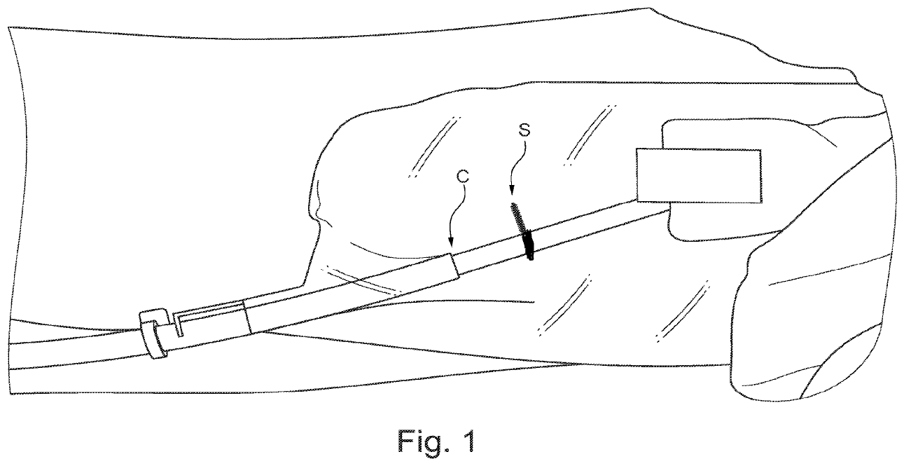

### DETAILED DESCRIPTION

Described below are devices to provide secure and reversible fixation of a cannula (or other flexible conduit) to the skin of a patient.

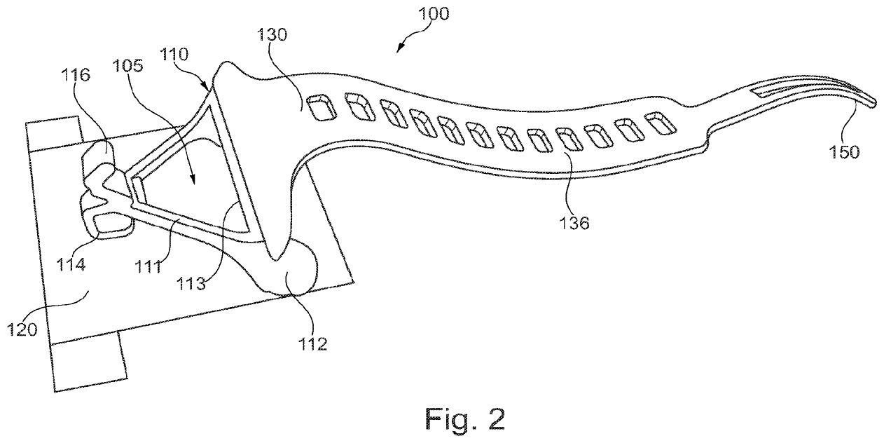

FIG. 2 shows a device 100 according to a first embodiment, in which the device secures a conduit that runs roughly parallel to the surface of the skin. Generally, the device 100 includes a stand or base 110, an adhesive element 120, and a strap 130. The base 110 includes a conduit-supporting portion with a conduit-supporting surface. In use, the conduit-supporting surface contacts the outside surface of a cannula (or other flexible conduit) placed on the base 110. The conduit-supporting portion includes first and second spacers or supports 112 (only one of which can be seen in FIG. 2). The supports 112 are positioned below the conduit-supporting surface to support the cannula. The adhesive element 120 is disposed on an underside of the base 110 to secure the device 100 to the skin of a patient. The supports 112 space the cannula from the adhesive element 120. The strap 130 has a first end attached to the base 110 and a second free end. The strap 130 extends from the base 110 adjacent to the conduit-supporting portion, and in use wraps around a cannula that is in contact with the conduit-supporting surface above the supports 112. In this way, once the strap 130 is made taut by a clinician, the cannula is frictionally engaged by the strap 130 and braced against the conduit-supporting surface and supported by the supports 112 of the base 110.

By comparison to one another, the base 110 is relatively rigid and the strap 130 is relatively flexible. The strap 130 is flexible so that a clinician is able to manipulate the strap 130 to wrap the cannula. The base 110 meanwhile acts as a rigid datum or foundation with which to anchor and stabilise the wrapped cannula.

In this embodiment, the base 110 is made of a VeroWhite resin and the strap 130 is made of a tear-resistant medical-grade silicone. In other embodiments, the strap 130 and base 110 may be integrally formed. Other rigid materials such as ABS plastic are also suitable for the base. The base and strap may be 3D printed or injection moulded.

The flexibility of the strap material also helps both to ensure that tension in the strap 130 does not affect the integrity of the cannula (for example, by crimping), even for thin-walled cannulae, and allows the device 100 to effectively secure cannulae (or other flexible conduits) of various shapes, including those with a non-circular profile.

Fastening elements or connectors are provided on the base 110 and the strap 130, which are interengageable or interlockable with each other to fasten the free end of the strap 130 to the base 110. In this way, once the clinician has tightened the strap 130 about the cannula, the respective fastening elements of the base 110 and the strap 130 can be interlocked to fasten the strap 130 in place, thereby securing the cannula to the device 100.

The supports 112 of the base 110 are spaced apart to allow the strap 130 to be received therebetween. In practice, the free end of the strap 130 can be passed through a gap between the supports 112, thereby providing the fastening element of the strap 130 access to the fastening element of the base 110.

In this embodiment, the base 110 has a single fastening element in the form of a hook 116, and the strap 130 has a series of fastening elements in the form of holes 136 which are sized to engage the hook 116. The holes 136 are arranged along the strap 130 between its first end and its free end.

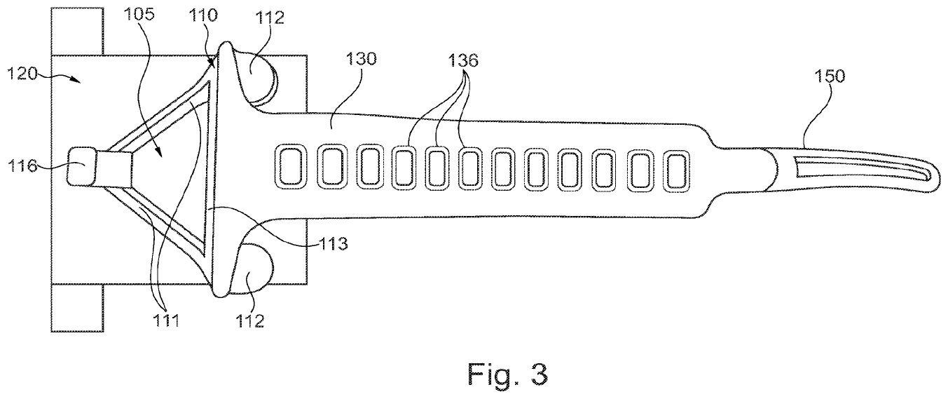

As can be seen more clearly in FIG. 3, the base 110 in this embodiment includes three load-bearing portions, or feet, arranged in a triangular formation. Two of these feet are positioned at a front or proximal side of the base (in the direction which in use is nearest the cannula), each forming a spacer or support 112, and a third foot 114 is positioned at a rear or distal side of the base (in the direction which in use is farthest from the cannula).

Each support 112 resembles a pedestal or ledge formed in the base, having an upper surface that forms part of the conduit-supporting surface which in use contacts the cannula. When placed on the conduit-supporting portion, the cannula is raised away from the skin of the patient by the supports 112, helping to prevent pressure sores from developing on the patient's skin. Importantly, by providing this elevation of the cannula, the supports 112 also facilitate a “side-docking” functionality of the device 100, by which additional devices 100 can be mounted to the cannula and removed from the cannula without the alignment of the cannula needing to be disturbed, as is described in more detail below.

In this embodiment, each support 112 is generally puck-shaped or disc-shaped, having a generally circular footprint, a curved upper surface, and a flat underside. The flat underside of each support 112 is attached to the adhesive element 120. In other embodiments, the supports 112 may take any practical shape. It is generally preferred, however, that the upper surface of each support 112 (which in this embodiment together form the conduit-supporting surface) is curved, and specifically concave, as in this embodiment, to better accommodate the curvature of the cannula. In this embodiment, the upper surface of each support 112 is curved in profile, arcing from a highest point at its distal end to a lower point at its proximal end. This curved profile forms an arcuate channel in the upper surface which is able to receive the curvature of the cannula surface to better seat the cannula on the conduit-supporting portion of the base 110.

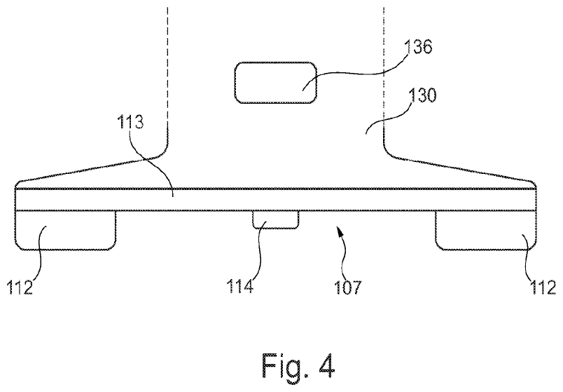

As described above, the supports 112 are spaced apart from each other, having a gap between them. The supports 112 are connected to one another in this embodiment by an overlying strut 113, which is positioned above each support 112 at their distal end. In this way, the supports 112 and the strut 113 define an aperture 107 in the base 110, as can be seen in FIG. 4. In use, the conduit thus rests against the strut 113 and on the upper surfaces (forming the conduit-supporting surface) of the supports 112. The gap between the supports 112 (and also the width and height of the aperture 107) is sized to permit the free end of the strap 130 to pass therethrough (that is, between the supports 112 and beneath the strut 113). In this embodiment, the supports 112 are spaced from each other by at least the maximum width of the free end of the strap 130 (that is, the maximum width in the region of the strap 130 having holes 136) so as to allow the strap 130 to pass between them without being rolled or furled at its edges. This allows a maximum surface contact to be achieved with the cannula across the width of the strap 130.

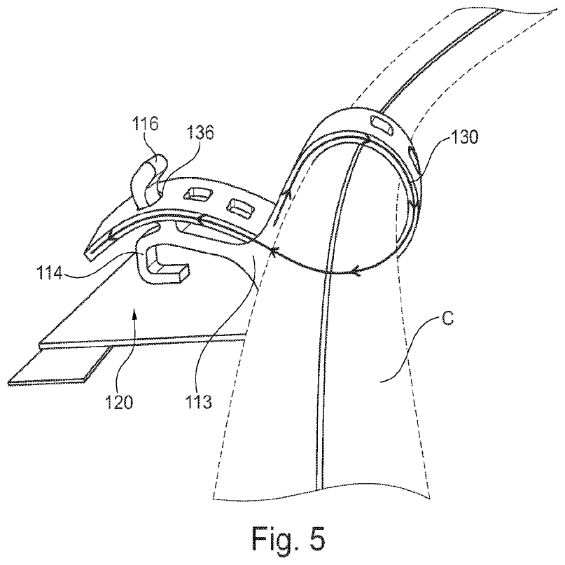

Referring now to FIG. 5, the rear foot 114 is longitudinally offset from the supports 112 and aligned centrally (that is mid-way) between the supports 112. In this embodiment, the rear foot 114 is generally C-shaped, having a curved upright portion at its rearmost side which joins an opposing pair of horizontal portions that project toward its foremost side. Like the supports 112, the rear foot 114 has a flat underside which is attached to the adhesive element 120 beneath. An upper side of the rear foot 114 connects to the hook 116. The hook 116 sits proud of the base 110, projecting upwardly from the rear foot 114. The hook 116 is longitudinally aligned with the holes 136 that are placed along the major axis of the strap 130. In this way, the hook 116 can be engaged with the holes 136 in the strap 130 while the longitudinal axis of the strap remains perpendicular to the longitudinal axis of a cannula being held in place in use. In this embodiment, the hook 116 is generally V-shaped, having two opposing portions which converge to form an apex at its rearmost side. In this configuration, the hook 116 and the rear foot 114 can together be considered to create a generally W-shaped profile. The W-shaped profile provides a particularly secure fixation between the hook 116 and hole 136, whereby the strap 130 is nestled or wedged in a nook between the hook 116 and the rear foot 114, helping to prevent the strap 130 from being accidentally released. However, the shapes of the rear foot and the hook 116 are not particularly limited and, in other embodiments, they can have any practical shape.

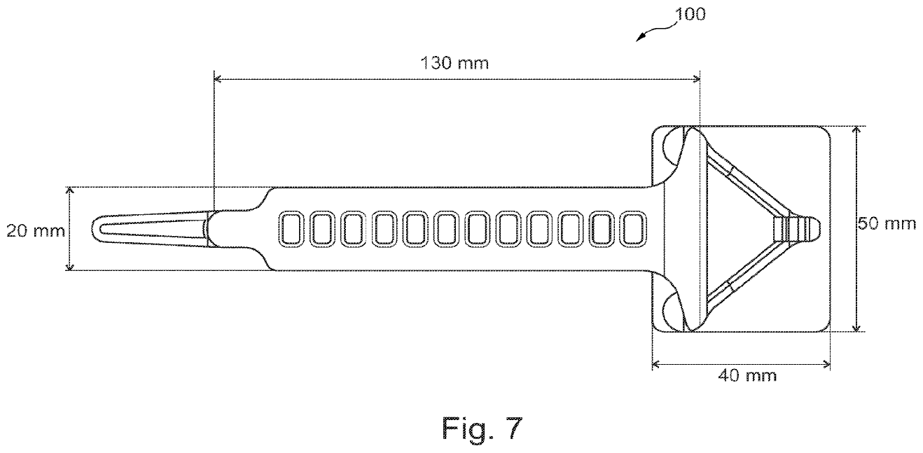

As can be seen in FIG. 7, the base 110 in this embodiment has a maximum width (measured from the outermost edge of one support 112 to the outermost edge of the other support 112) of 50 mm, and a length (measured in the direction of the longitudinal axis of the strap, from the proximal edge of the supports 112 to the distal edge of the foot 114) of 40 mm. It is preferred that the distance between the midpoints of the two front feet is from 15 mm to 40 mm, and that the distance from the rear foot to a point midway between the midpoints of the front feet is from 15 mm to 40 mm.

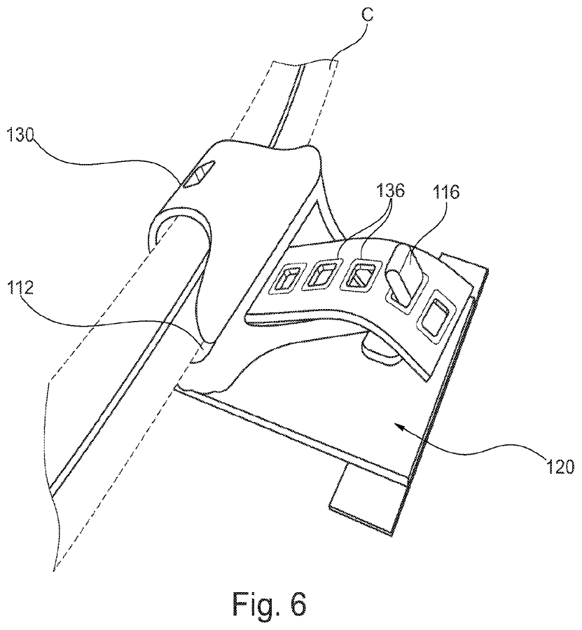

Returning to FIG. 2, the base 110 also includes a pair of further struts or bars 111, which connect each of the supports 112 with the rear foot 114. In this way, the bars 111 and strut 113 form a triangular opening 105 in the base 110, the hook 116 being positioned at a distal apex of the triangular opening 105, the other apices being located at the supports 112. The opening 105 is in communication with the gap between the supports 112 (and, therefore, also the aperture 107 formed between the supports 112 and the strut 113). The opening 105 thereby allows the free end of the strap 130 (once looped over the cannula and threaded between the supports 112) to be passed from beneath the strut 113 to the upper side of the base 110, where it is able to meet the hook 116. In other words, through the aperture 107 and the opening 105 in the base, the strap can be looped around the cannula in the direction away from the hook 116 and return to meet the hook 116 at the opposite side of the base 100 to the cannula. This over-and-under arrangement, as is shown in FIGS. 5 and 6, allows the strap 130 to wrap the cannula completely, so that the strap 130 engages substantially the entire circumference of the cannula, maximising the contact and therefore the frictional engagement with the cannula. Further, this arrangement enables the device 100 to be installed, fastened to the cannula, and removed from the patient's skin all from a single side of the cannula. This is the “side-docking” functionality of the device 100 as referred to above, which avoids the cannula needing to be manipulated out of its alignment (e.g. raised over a projecting portion of the device) when the device is installed or removed.

The feet of the base 110 (i.e. the supports 112 and the rear foot 114) also provide the base 110 with a non-solid footprint. That is, the underside of the base 110 makes contact with the adhesive element 120, and therefore transfers the weight of the device 100 and the cannula to the skin, at multiple points, between which there are regions of open space. This footed arrangement means that the device 100 can be stably mounted on various different contours of the patient's skin, which allows the device 100 to be secured at various positions on the body. For example, in ECMO procedures cannulae may be inserted at sites at either the thighs or the neck of a patient. Further, in this embodiment, the points of contact between the base 110 and the adhesive element 120 (i.e. the locations of the feet) are positioned at the extremities of the base 110, maximising the stability of the base 110. The tripod configuration of the illustrated embodiment is particularly advantageous in these respects. However, in other embodiments, the base 110 can have two, four, or five or more feet. It will be appreciated that this non-solid footprint, in which multiple feet are provided, and in particular the tripod arrangement of the base 110 may be isolated from the other features of the device and is independently advantageous.

Similarly, while in the preferred embodiment illustrated, the feet correspond to the apices of the opening 105, the shape of the opening 105 need not be defined by the feet. In other embodiments the opening 105 in the base may take any practical shape, for example a slot, or hole of other shape. If the fastening point of the base 116 is provided above the aperture 107 formed between the supports 112, then as long as the strap 130 can pass through the base 110 to reach it, the opening 105 can take any form that provides for this.

As described above, the strap 130 has a first end attached at the base 110 and a second free end, the first end being positioned at a point on the base 110 adjacent to the supports 112. In particular, in this embodiment, the strap 130 extends from the strut 113 which overlies and connects the supports 112. The first end of the strap 130 includes a flared portion, being widest where it connects to the base 110 at the strut 113. Thereafter, along the majority of its length, the strap 130 has a constant width, coming to a rounded end. At the first end, the strap 130 extends initially upward from the base 110.

The strap 130 in this embodiment is an elongate tongue, having a relatively wide and flat profile. As can be seen in FIG. 7, the strap 130 has a length (from its first end to its second end) of 130 mm, and a width (across its breadth in the region of the holes 136) of 20 mm. The breadth of the strap 130 provides maximal surface contact with the cannula so as (when taut) to best distribute pressure over the cannula surface (helping to prevent excess concentration of tension that might otherwise possibly deform or crimp the cannula). The holes 136, which act as the fastening elements of the strap 130, are spaced lengthways along the strap 130 and aligned with one another. In this embodiment, the strap 130 includes twelve holes 136 which are spaced at equal increments along the longitudinal axis of the strap 130. The plurality of holes 136 allows the device 100 to accommodate a variety of sizes of cannulae (or other flexible conduits). For example, in the embodiment shown, cannulae of diameters from 3 mm to 16 mm can be secured by the strap 130. The holes 136 are positioned at regular intervals along the strap 130 to accommodate standard sizes of cannulae within this range.

As described above, the base 110 is made of a comparatively rigid material and the strap 130 is made of a comparatively flexible material. In this embodiment, the material of the strap 130 (in this case a tear-resistant silicone) also provides a non-slip surface. In other words, the material of the strap 130 provides a beneficially high coefficient of static friction between the surfaces of the strap and the cannula. Although preferred, the non-slip surface is, of course, not necessary in all embodiments.

In this embodiment, at the free end of the strap 130 there is a tail 150. The tail 150 protrudes from the free end of the strap 130 and is narrower than the majority of the strap 130 (in particular, narrower than the region of the strap 130 along which the holes 136 are disposed), making it easier for the clinician to thread the strap 130 through the gap between the supports 112 (in other words, through the aperture 107) and up through the opening 105. The tail 150 is also relatively more rigid than the strap 130, providing a firm point of contact with which the clinician can manipulate the strap 130. In this embodiment, the tail 150 is made of VeroWhite resin (as is the base 110). The relative rigidity of the tail 150 may also be achieved by increased thickness compared to the remainder of the strap 130. In this way, the tail 150 better allows the clinician to grip and guide the strap 130, with minimal movement of the cannula. The tail 150, in this embodiment, is also curved in shape, having an arched profile which further improves its grippability and manipulability. In other embodiments, a similar resting curvature (that is, a curved profile when not under load) can be incorporated over a portion or the entirety of the strap 130.

This resting curvature can be achieved, for example, by forming the strap 130 from two sheets of silicone, one of which is longitudinally pre-stressed such that a portion of the strap 130 is biased towards forming a curve. Alternatively, the resting curvature can be achieved by moulding the strap with an inherent curve. In another embodiment, the resting curvature is achieved using a bistable structure in which the resting curve is activated as the strap is initially deformed. One way to do this is by the strap having a non-uniform thickness and/or density. For example, the thickness and/or density of the strap may be varied on one side, thereby causing the strap to fall into the desired curvature. The skilled person will be aware of other ways of achieving a desired resting curvature. The strap in other embodiments of the device does not have a tail.

The strap 130 can also incorporate materials or surface preparations that exhibit a visual change under specific tensional loads to give a visual indication of over-tightening. For example, the visual change may be provided by a moiré pattern on the surface of the strap 130 which provides an indication of the degree of deformation of the strap 130. Similarly, the visual change may be astress-dependent colour change, which can be achieved, for example, by the incorporation of existing technologies which mimic the microstructure on the surface of Morpho butterfly wings. An example is the Morpho structure as described by Benjamin Miller et al. of Massachusetts Institute of Technology, and featured in: McDougal et al. (2019) “Biological growth and synthetic fabrication of structurally colored materials.” Journal of Optics 21, 073001.

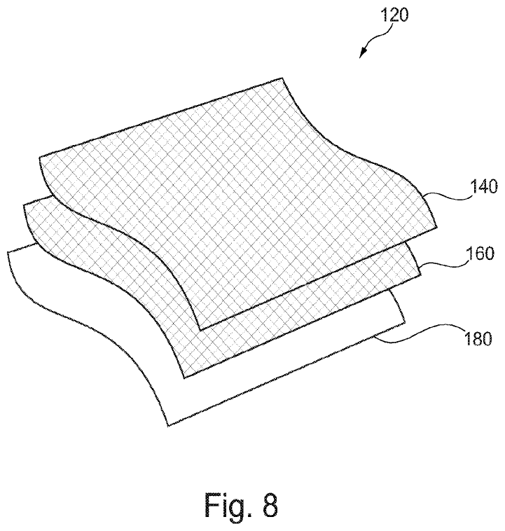

With reference to FIG. 8, the adhesive element 120 in this embodiment is a kinesiology tape that generally includes three layers: a membrane 140, an adhesive layer 160, and a releasable liner 180. The membrane 140 is a fabric layer or carrier, the upper side of which is connected to the underside of the base 110 using permanent adhesive. The adhesive 160 is disposed on and bonded to an underside of the membrane 140 and, in use, provides adhesion of the device 100 to the skin of the patient. The release liner 180 is a peelable sheet which is removably disposed on the adhesive layer 160, temporarily covering an underside of the layer of adhesive 160 before the device 100 is placed and adhered against the patient. In this embodiment, the release liner 180 is a paper sheet, but it can otherwise be made of plastic or any other suitable material. Some embodiments have no release liner 180. In some embodiments, the adhesive element 120 may not include a membrane 140 or liner 180, an adhesive instead being deposited directly on the base 110. In other embodiments, any suitable form of adhesive or fixative component may be used as the adhesive element 120.

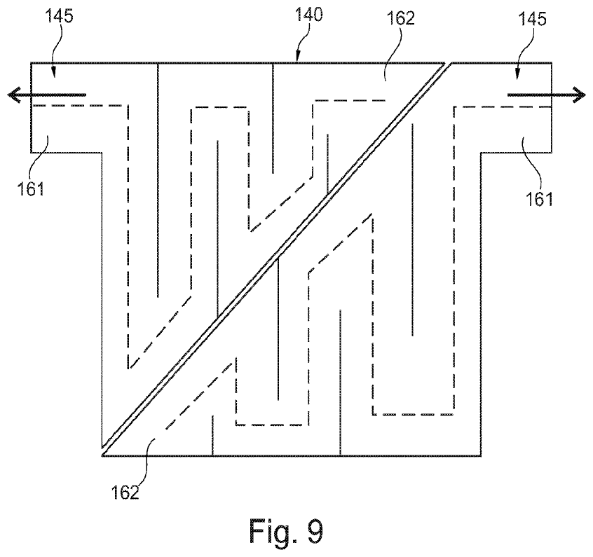

With reference to FIG. 9, the adhesive layer 160 in this embodiment is deposited on the underside of the membrane 140 in a particularly advantageous pattern. With this pattern, the membrane 140 is bifurcated along a central boundary line, the adhesive layer 160 being disconnected between one side of this boundary line and the other. The boundary line bisects the underside of the membrane 140 along a diagonal with respect to the longitudinal axis of the device 100. On either side of this line, the adhesive 160 is deposited in a zigzag or serpentine pattern between a first end 161 and a second end 162. At the first end 161 of each serpentine section, the membrane 140 has a pull tab 145. Each pull tab 145 is bonded to the adhesive layer 160 at the corresponding first end 161. The pull tabs 145 protrude from beneath the main body or extent of the membrane 140 and are separable from the main body or extent of the membrane 140. In this way, the pull tabs 145 can be pulled by a clinician to release and peel away the adhesive layer 160 from the membrane 140. When the pull tab 145 is pulled by the clinician, the adhesive 160 is progressively released, first at its first end 161 and gradually to its second end 162, as is shown in FIG. 9. In this way, the adhesive layer 160 can be separated from the membrane 140 to remove the device 100 from the skin of a patient, with minimal movement of the device as a whole.

The sectioning of the adhesive 160 on the membrane 140 enables the adhesive force to be more easily overcome when desired (i.e. during removal), while maintaining the strength of adhesion of the adhesive element 120 as a whole. In particular, the serpentine configuration of the adhesive layer 160 thereby enables the clinician to remove the adhesive element 120 from the skin of a patient by pulling the tabs 145 of the membrane 140 generally parallel to the patient's skin, avoiding undesirable upward movement or rotation of the device 100. This is enormously beneficial in maintaining the precise orientation of the device 100 during its removal. Further, the gradual release of the adhesive 160 smoothens removal, reducing the “pull” or “drag” of the adhesive 160 against the patient's skin when peeled away. This further lessens the possibility that the precise alignment of the cannula is accidentally disturbed and also reduces patient discomfort.

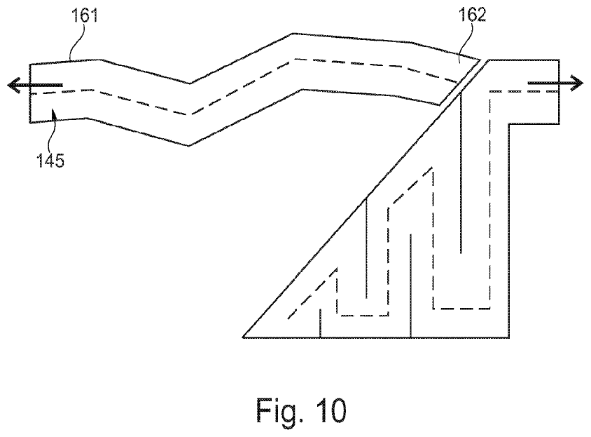

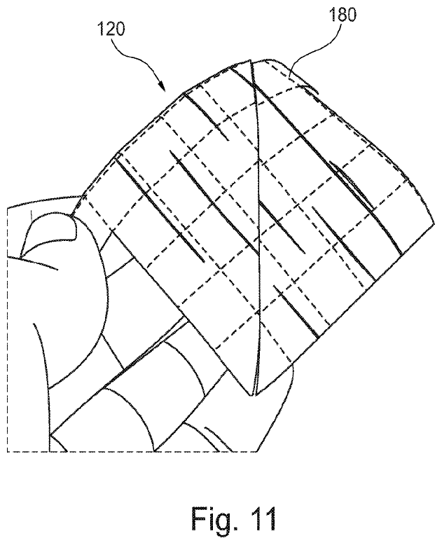

In this embodiment, the release liner 180 covers the entirety of the underside of the membrane 140 and is sectioned to mirror the pattern of the adhesive 160, as is shown in FIG. 10. In particular, in the embodiment shown, the membrane 140 is a continuous sheet (except for the pull tabs 145 which are separate) over which an initially continuous layer of adhesive 160 is spread. A series of “cuts” are then simply scored into the adhesive layer 160 through the paper release liner 180 to achieve the sectioned pattern as shown.

Processes of deploying, securing, removing, and replacing the device 100 in practice are now described.

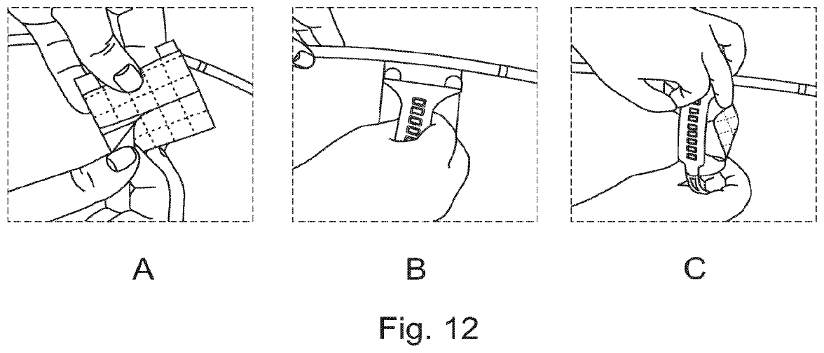

In practice, an initial device 100 is first deployed. This process involves placing the device 100 adjacent to the cannula (or other flexible conduit) to be secured and adhering the device 100 to the skin of the patient with the adhesive element. The method involves the following steps, as shown at FIG. 12:
A. The clinician first upturns the device 100 and peels away a section of the release liner 180 from the adhesive element 120, revealing a proximal portion of the adhesive layer 160.
B. The device 100 is then slid into place adjacent to the cannula, so that the supports 112 are beneath the cannula, and the exposed portion of the adhesive layer 160 makes contact with the skin below.
C. The clinician then removes the remaining distal section of the release liner 180 and lightly presses the exposed adhesive 160 to the skin, sticking the device 100 in place.

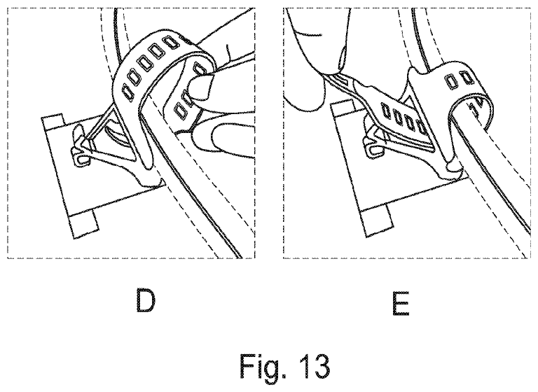

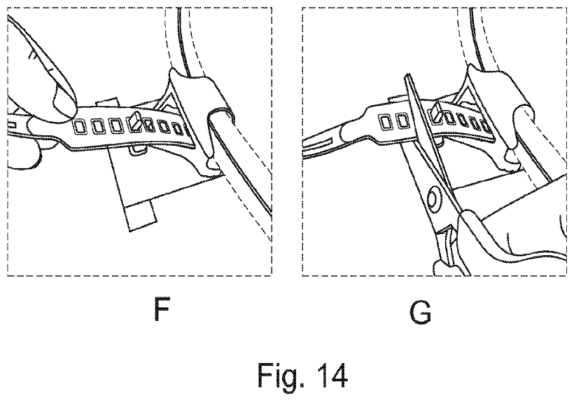

At this stage, the device 100 is secured to the patient's skin and abuts the cannula, which rests on the conduit-supporting surface on the supports 112 but is generally free to move back or forth in the direction of its longitudinal axis and upward from or proximally of the base 110. The device 100 is then fastened to secure the cannula (or other flexible conduit) to the base 110. This process involves wrapping the cannula with the strap 130 and engaging the respective fastening elements of the base 110 and the strap 130 with one another to constrain any movement of the cannula. The method for fastening the device 100 to the cannula in this way involves the following steps, as shown at FIGS. 13 and 14:
D. The device 100 is in position adjacent to the cannula, with the conduit-supporting surface on the supports 112 abutting the lower surface of the cannula. The clinician then optionally grasps the tail 150 to guide the strap 130. The strap 130 is looped over the cannula and threaded beneath the strut 113 and through the gap between the supports 112.
E. The strap 130 is then passed through the opening 105 to the upper side of the base 110 and pulled taut. When pulled taut, the strap 130 is in frictional engagement with the cannula, which is braced against the conduit-supporting portion, supported by the supports 112.
F. A hole 136 of the strap 130 is then engaged or interlocked with the hook 116 at the distalmost apex of the opening 105. This fastens the free end of the strap 130 to the base 110, maintaining the engagement between the taut strap 130 and the cannula. In this configuration, the strap 130 and the rigid base 110 constrain the cannula from moving in every degree of freedom (that is, the cannula is made fast so that it is unable to shift by translation or rotation in any direction).
G. An excess length of the free end of the strap 130 can then optionally be removed with scissors.

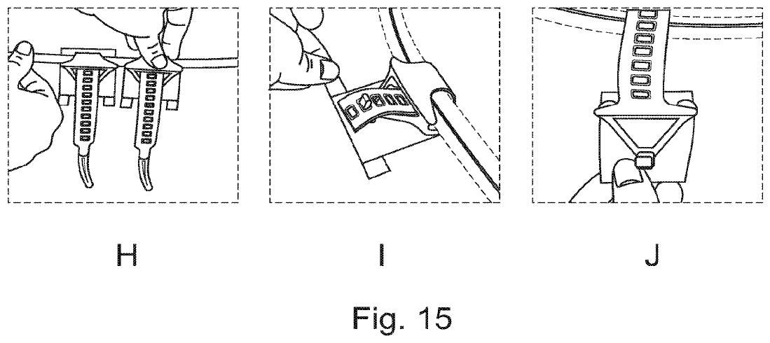

When it is time to remove the device 100, this process can be simply and easily reversed, even by a single clinician. In an ECMO procedure, for example, the device 100 may be swapped out for a new one after it has been in situ for 5 or 6 days. The process of introducing an additional device 100 and removing that which is already in situ involves the following steps, as shown at FIG. 15:
H. A second device 100 is placed against the cannula beside the first device 100 which is already secured in situ. The second device 100 is secured in the fashion as described above. It will be appreciated that, beneficially, the second device 100 can be slid or “side-docked” into place against, and fastened to, the cannula without disturbing the alignment of the cannula.
I. The first device 100 is then removed. The clinician initiates the removal by pulling each of the pull tabs 145 of the adhesive membrane 140 away from the base 110 and generally parallel to the skin of the patient, thereby releasing the adhesive 160 of the adhesive element 120 from the skin.
J. The strap 130 of the first device 100 is then disengaged from the hook 116 and the entire device 100 may simply be slid away from the cannula, generally parallel to the surface of the patient's skin.

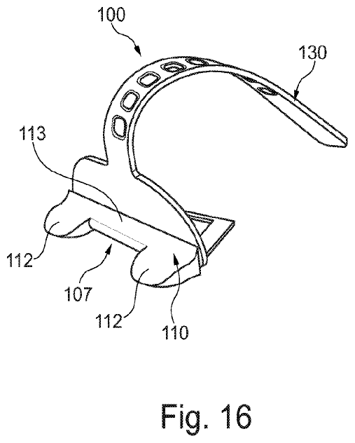

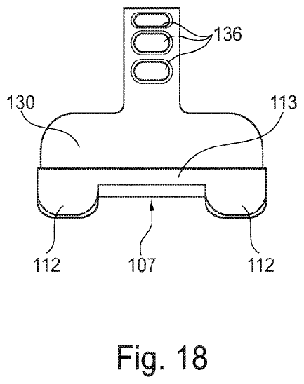

FIGS. 16, 17, and 18 show a further embodiment of the device 100. With reference to FIG. 16, this embodiment 100 similarly includes a base 110 and a strap 130. However, this device 100 is shown without an adhesive element. It will be appreciated that any suitable adhesive element (as described above) can be disposed on an underside of the base 110 to secure the device 100 to the skin of a patient. The structure of the base 110 and the strap 130 largely mirror those as described above.

By comparison to the first embodiment, the base 110 in this embodiment has a relatively shallower profile, the supports 112 and the rear foot 114 being shorter, the strap 130 being thinner, and the space beneath the strut 113 shallower. This embodiment achieves a comparable function to the first embodiment, with minimal material. The supports 112 are again generally disc-shaped. However, in profile, the supports 112 resemble a goldfish; that is, in profile, each support 112 narrows to its proximal edge and broadens to its distal edge, having a pinched middle section therebetween that produces an arch in the upper and the underside of the support 112. As a result, the concavity of the conduit-supporting surface is more pronounced in this embodiment. Further, the pointed profile of the supports 112 toward their proximal edge in this embodiment aids in introducing the device 100 beneath a cannula (especially so in the case that there is already a first device 100 in situ). As above, the supports 112 are each connected to the foot 114 by a respective bar 111 of a pair of bars 111. In this embodiment, each bar 111 is arched upward, and is formed so as to allow for some flexion upwards or downwards. The shallower profile of this embodiment allows for improved distribution of compressive load when the whole device and conduit are pressed down on (for example, in the event that a patient is ‘proned’ temporarily), while the flexural compliance of the bars 111 still allows the device to mate to various anatomies, accommodating a variety of curvatures.

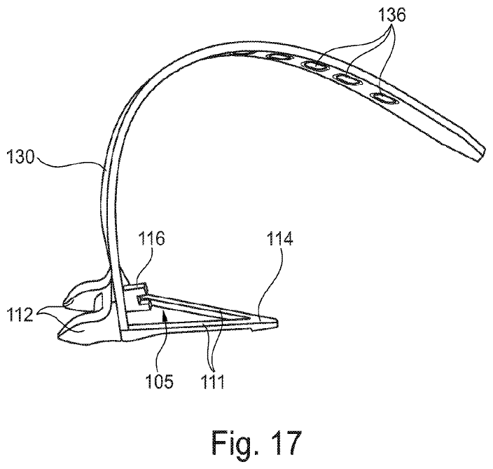

The primary distinction between the device 100 in this embodiment, compared with the previous embodiment, is the configuration of the fastening element on the base 116. As can be seen in FIG. 17, the hook 116 of the first embodiment is replaced with a clasp or clip 116. The clip 116 is generally C-shaped, having a pair of opposing prongs which are configured to interlock with a portion of the strap 130 between a pair of holes 136. Rather than being positioned at the rear foot as the hook 116 of the previous embodiment is, the clip 116 is disposed on the reverse of the strut 113. That is, the clip 116 is disposed on the strut 113 at an opposite side of the strut 113 to the supports 112 and adjacent to the opening 105. In other words, the clip 116 is positioned at an interior side of the opening 105 (rather than at its distalmost apex as the hook 116 in the previous embodiment is). The point at which the strap 130 extends from the base 110 (as before) is positioned between the supports 112 and the fastening element of the base 116. In this arrangement, the free end of the strap 130 is again able to be looped over a cannula that is in contact with the conduit-supporting surface, passed through the gap between the supports 112, and, from beneath the strut 113, up through the opening 105, whereby the holes 136 are able to meet the clip 116.

The second embodiment of the device 100 is used (that is deployed, fastened, removed, and replaced) in practice in the same way as the first embodiment.

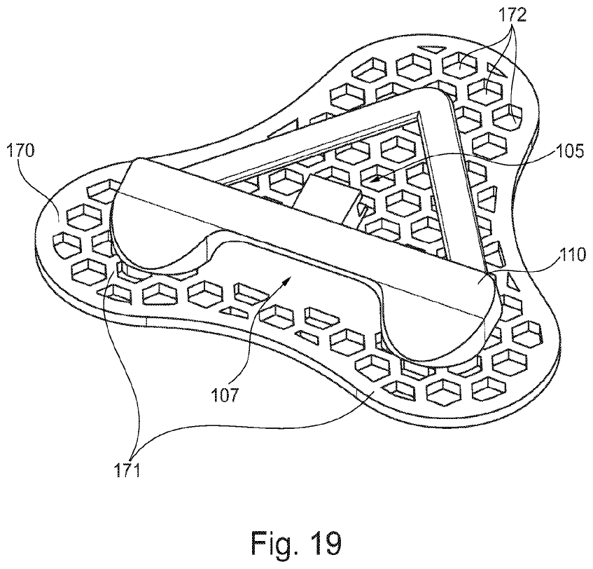

FIG. 19 shows another embodiment of the device 100. This embodiment is a variation on the second embodiment of FIGS. 16-18, in which the base 110 is affixed to a flexurally-compliant pad 170. Although the strap 130 and the adhesive element 120 are not shown in FIG. 19, those described above in respect of earlier embodiments are equally applicable to this embodiment. The adhesive element 120 is located beneath and affixed to the underside of the pad 170. The strap 130 extends from the base 110 as in the previous embodiments.

The pad 170 is a substantially planar mesh or web comprising an abundance or multiplicity of tessellated cells 172. In this embodiment, each cell 172 is hexagonal, forming a honeycomb mesh. The mesh is bounded by an exterior band or perimeter. The meshed structure of the pad 170 provides a combination of axial stiffness (along the plane of the pad 170, parallel to the skin) and compliance in flexion (perpendicular to the plane of the pad 170).

The flexurally-compliant pad 170 enables additional distribution of compressive loads, which may emerge, for example, when patients are proned or rolled temporarily. Once the device is affixed to the skin (with the adhesive element 120) any deflection of the pad 170 will not compromise the stability of the device and therefore the conduit it holds. The flexural compliance of the pad 170 maximises conformity with the skin surface. The flexible and therefore compliant pad 170 retains the versatility provided by the tripod arrangement of the base, while additionally providing a conforming contact surface with the skin. This conforming surface allows the adhesive to work to greater effect. The meshed structure allows for local deformation, and therefore greater compliance than a continuous sheet of material (e.g. plastic).

In this embodiment, the pad 170 has a trefoil shape. That is, the pad 170 has three lobes 171. Each lobe 171 is arranged beneath and surrounding a respective foot of the base 110. The lobed shape of the pad 170 further improves compressive load distribution (and therefore minimises the risk of pressure sores) when the device is pressed down during patient-proning for example, by providing a route for compressive forces to be transmitted from the three rigid feet of the base 110 over a wider area over the surface of the skin.

A solid area (that is, a portion of the pad 170 without cells 172) is located between the two front feet 112, beneath the strut 113. This solid area allows for sliding of the strap 130 against the surface of the pad 170, for smoother insertion of the strap 130 through the aperture 107.

In this embodiment, the pad 170 is 3D printed from an ‘Agilus30’ polymer which is somewhat flexible, but stiff enough to prevent unacceptable movement of the conduit relative to the skin. The degree of axial stiffness required is reduced by the fact that the skin is inherently compliant. Power-matching in the kinematic chain means that there is limited benefit in making the flexible pad much stiffer than the anatomy itself; some axial compliance, therefore, does not hinder performance compared to the fundamental limits of the substitial anatomy. The combination of flexural compliance and relative axial stiffness is provided through material selection and the effective embedding of joints (i.e. intersections between the cells of the mesh).

Once the device is secured to the skin, the kinematic chain between the skin, device, and conduit is rigid since the base 110 is rigid and the aforementioned flexion of the pad 170 is constrained to the skin by the action of the adhesive.

In this embodiment, the pad 170 is printed (from the Agilus 30 polymer) continuously with the rigid base (made of a different polymer), and bonded to the membrane 140 of the adhesive element 120. In other embodiments, the pad can be integrated into the membrane 140, with other aspects of the adhesive and liner implemented as described previously.

Of course, the skilled person will appreciate that many modifications may be made to the above-described embodiments.

For instance, while FIG. 7 shows the dimensions of the base 110 and the strap 130 in the preferred embodiment, it will be appreciated that these dimensions are provided by way of example only and any suitable size and proportions may be used.

Further, it is noted that, although in the embodiments shown the conduit-supporting surface is formed of two parts (a part being disposed on an upper surface of each of the supports 112), other arrangements of the conduit-supporting portion are contemplated. In particular, the conduit-supporting surface need not be confined to or coterminous with the supports 112. For example, the conduit-supporting surface can be a continuous or singular ledge formed in the base 110, separate from and/or extending between the supports 112. It will be appreciated that, in embodiments where the conduit-supporting surface is continuous, it is preferable that the conduit-supporting surface includes a recess or space (specifically at least in a region between the supports 112 and beneath the strap 130) to accommodate the strap 130. In allowing a portion of the strap 130 to be nestled between first and second parts of the conduit-supporting surface, such a space maximizes the proportion of the strap 130 which is able to contact the cannula, improving the engagement between the strap 130 and the cannula. The most preferred embodiments, such as those illustrated, allow substantially a complete circumference of the cannula to be engaged by the strap 130 in this way.

Similarly, it will be appreciated that, although in the illustrated embodiments the aperture 107 is defined by the supports 112 and the overlying strut 113 which connects them, the configuration of the aperture 107 need not be so limited, and can take any practical form. For example, in other embodiments, the aperture 107 can be a slot formed in the base 110, separate to and independent of the strut 113.

In addition, in other modifications any suitable interengageable connectors can be used as the respective fastening elements. For example, in some embodiments hook-and-loop connectors, snap fasteners, or magnetic connectors may be used. Similarly, it is not necessary that the base 110 include a single fastening element and the strap 130 include multiple fastening elements as is illustrated; the base 110 may have multiple fastening elements; the strap 130 may have a single fastening element. Any practical combination or distribution of fastening elements between the base 110 and strap 130 can be used. Further, in the illustrated embodiment, the base 110 has a male fastening element and the strap 130 has a plurality of female fastening elements. However, in other embodiments, the male and female components may be reversed. In some embodiments, each fastening element need be neither male nor female (in the case, for example, that magnetic connectors or even adhesive are used).

The described embodiments provide improvements in being able reversibly to secure a flexible conduit, in particular a cannula to the skin of a patient. As well as being useful in ECMO procedures, a number of additional needs for different clinical use cases are met. The flexibility of the described devices makes it equally applicable to similar challenges in other clinical processes where flexible conduits for electrical signals, gases, and fluids are affixed to the patient, and in general cable and line management for patients. These other applications include, but are not limited to the fixation of: endotracheal tubes, naso-gastric tubes, central venous catheters, arterial line catheters, Swan Ganz catheters, indwelling oesophageal Doppler devices, surgical drain catheters, surgical manometers, epidural catheters, local anaesthetic catheters, suprapubic catheters, and dialysis tubing. The impact of conduit dislodgement in these applications ranges from patient discomfort to clinical emergencies and mortality.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosures in United Kingdom patent application GB 2210151.3, from which this application claims priority, and in the accompanying abstract are incorporated herein by reference.

### REFERENCES

McDougal et al. (2019) J. Opt. 21, 073001

1. A device for reversibly securing a flexible conduit to the skin of a patient, including:
a rigid base having a conduit-supporting portion with a conduit-supporting surface;
an adhesive element disposed on an underside of the base to secure the device to the skin; and
a flexible strap extending from the base adjacent to the conduit-supporting portion; wherein:
the base has a first fastening element and the strap has a second fastening element, the first and second fastening elements being interengageable with each other;
the conduit-supporting portion includes first and second spacers below the conduit-supporting surface; and
the spacers are spaced from one another to receive the strap therebetween, providing the second fastening element access to the first fastening element therethrough.

2. A device as claimed in claim 1, wherein the strap extends from the base at a position above the conduit-supporting portion.

3. A device as claimed in claim 1, wherein the conduit-supporting surface includes first and second portions which are spaced from each other to accommodate the strap therebetween.

4. A device as claimed in claim 3, wherein a respective portion of the conduit-supporting surface is at an upper side of each spacer.

5. A device as claimed in claim 1, wherein the base has a non-solid footprint.

6. A device as claimed in claim 1, wherein the base includes multiple feet.

7. A device as claimed in claim 1, wherein the base has three feet.

8. A device as claimed in claim 6, wherein each foot is positioned at an extremity of the base.

9. A device as claimed in claim 6, wherein the first and second spacers form first and second feet of the base.

10. A device as claimed in claim 1, wherein the first fastening element is positioned on the base at a point higher than or level with the position at which the strap extends from the base.

11. A device as claimed in claim 1, wherein the strap has multiple fastening elements, each of which is interengageable with the first fastening element on the base.

12. A device as claimed in claim 1, wherein the strap includes a portion having a resting curvature.

13. A device as claimed in claim 1, wherein the thickness of the strap is non-uniform.

14. A device as claimed in claim 13, wherein the thickness of the strap varies on one side.

15. A device as claimed in claim 1, wherein the density of the strap is non-uniform.

16. A device as claimed in claim 15, wherein the density of the strap varies on one side.

17. A device as claimed in claim 1, wherein the strap includes a material or surface preparation that exhibits a visual change under specific tensional loads to give a visual indication of over-tightening.

18. A device as claimed in claim 1, additionally including a flexurally-compliant pad disposed between the base and the adhesive element, the pad having a multiplicity of cells.

19. A method of reversibly securing a flexible conduit to the skin of a patient including:
adhering a device as claimed in claim 1 to the skin of a patient;
supporting a conduit on the conduit-supporting surface;
wrapping the strap around the conduit;
feeding the strap between the spacers; and
engaging the first and second fastening elements.

20. A method as claimed in claim 19, including removing excess length at the free end of the strap.
